SEQUENCE LISTING (3 sequences)

SEQ ID NO: 1 - DNA, 40 bp, Artificial Sequence
  Aptamer 1
cgtgcagtacgccaacctttctcatgcgctgcccctctta

SEQ ID NO: 2 - DNA, 40 bp, Artificial Sequence
  Aptamer 2
cgcatgccaaacgttgcctcatagttccctccccgtgtcc

SEQ ID NO: 3 - DNA, 40 bp, Artificial Sequence
  Control Line DNA Strand
ggacacggggagggaactatgaggcaacgtttggcatgcg

(19) United States
(12) Patent Application Publication
(10) Pub. No.: US 2022/0134340 A1
(43) Pub. Date: May 5, 2022

(54) PAPER-BASED LOW-COST MICROFLUIDIC DEVICES FOR AUTOMATIC MULTISTEP PROCESSES

(71) Applicant: The Texas A&M University System, College Station, TX (US)

(72) Inventors: Dandan Tu, College Station, TX (US); Gerard L. Coté, College Station, TX (US); Samuel Mabbott, College Station, TX (US); John Dean, College Station, TX (US)

(73) Assignee: The Texas A&M University System, College Station, TX (US)

(21) Appl. No.: 17/512,787

(22) Filed: Oct. 28, 2021

Related U.S. Application Data
(60) Provisional application No. 63/108,581, filed on Nov. 2, 2020.

(51) Int. Cl. B01L 3/00; C12N 15/115; G01N 33/68; G01N 33/53; G01N 21/65

(52) U.S. Cl. CPC B01L 3/502746; C12N 15/115; B01L 3/502707; B01L 3/50273; B01L 3/567; G01N 33/6893; B01L 2400/06; G01N 21/658; C12N 2310/16; B01L 2300/126; B01L 2400/0677; B01L 2300/0816; G01N 33/5308

(57) ABSTRACT

In an embodiment, the present disclosure pertains to a microfluidic device composed of a substrate having an inlet region and a first storage region, a fluid transporting channel in fluid communication with the inlet region, an expandable component in fluid communication with the fluid transporting channel and coupled to a movable arm, and a fluid transporting region coupled to the movable arm and operable to be moved in a horizontal direction to the fluid transporting channel to thereby form fluidic contact between the inlet region and the first storage region upon expansion of the expandable component. In an additional embodiment, the present disclosure pertains to a method of fluid flow utilizing a microfluidic device of the present disclosure.

### CROSS-REFERENCE TO RELATED APPLICATIONS

This application claims priority to U.S. Provisional Patent Application No. 63/108,581, filed on Nov. 2, 2020. The entirety of the aforementioned application is incorporated herein by reference.

### STATEMENT REGARDING FEDERALLY SPONSORED RESEARCH

This invention was made with government support under 1648451 awarded by the National Science Foundation. The government has certain rights in the invention.

### BACKGROUND

A microfluidic paper-based analytical device (μPAD) is a cost-effective platform to implement assays, especially for point-of-care testing. Developing μPADs with fluidic control is important to implement multi-step assays and provide high sensitivities. However, current localized delays in μPADs have limited ability to decrease the flow rate. Additionally, existing μPADs for automatic multi-step assays are limited by their need for auxiliary instruments, their false activation, or their unavoidable tradeoff between available fluid volumes and temporal differences between steps.

### SUMMARY

In an embodiment, the present disclosure pertains to a microfluidic device composed of a substrate having an inlet region and a first storage region, a fluid transporting channel in fluid communication with the inlet region, an expandable component in fluid communication with the fluid transporting channel and coupled to a movable arm, and a fluid transporting region coupled to the movable arm and operable to be moved in a horizontal direction to the fluid transporting channel to thereby form fluidic contact between the inlet region and the fluid storage region upon expansion of the expandable component.

In an additional embodiment, the present disclosure pertains to a method of fluid flow. In general, the method includes one or more of the following steps of: (1) receiving a first fluid at an inlet region on a substrate; (2) receiving a second fluid at a storage region on the substrate; (3) flowing the first fluid through a fluid transporting channel on the substrate in fluid communication with the inlet region; (4) actuating a fluid transporting region coupled to a movable arm operable to be moved in a horizontal direction parallel to the fluid transporting channel via expansion of an expandable component in fluid communication with the fluid transporting channel; and (5) flowing the second fluid through the fluid transporting channel.

### DESCRIPTION OF THE DRAWINGS

FIG. 1A depicts a microfluidic device according to an aspect of the present disclosure.

FIG. 1B depicts a method of fluid flow according to an aspect of the present disclosure.

FIG. 2 illustrates a schematic representation of a microfluidic paper-based analytical device (μPAD) with a horizontal motion mechanical valve.

FIG. 3 illustrates a schematic representation of the μPAD with a mechanical valve and a localized dissolvable delay for a multi-step assay. Components and dimensions of the localized dissolvable delay portion of the μPAD are shown in the blow-up detail on the right.

FIG. 4A illustrates a comparison of the flow time of the fluid front to reach 40 mm on a μPAD without a dissolvable delay, a μPAD with a localized dissolvable delay made of 3 L of 0.6 g/mL sucrose, and a μPAD with a localized dissolvable delay made of 3 μL of 0.6 g/mL sucrose with 1.0 g/mL fructose. All the localized dissolvable delays were located at the 10 mm position (^{†}significantly different; p<0.05). FIG. 4B shows the column data is the flow time on the μPADs with localized dissolvable delays made of different volumes of 0.6 g/mL sucrose with 1.0 g/mL fructose, and the scatter data is the length of the sugar region. All the localized dissolvable delays were located at the 10 mm position. FIG. 4C shows an image of the μPADs with localized dissolvable delays made with 2.5 μL of 0.6 g/mL sucrose and 1.0 g/mL fructose at different positions (light grey area shifted from left to right as you go from bottom to top). FIG. 4D shows flow time for the fluid front to reach 40 mm on the hydrophilic channel of the μPADs (*significantly different compared with delays located at different positions; p<0.05).

FIGS. 5A1, 5A2, and 5A3 illustrate a schematic representation of using the μPADs with a mechanical valve to finish a two-step process automatically. FIG. 5B shows height change of the actuator and FIG. 5C shows horizontal movement of the arm in a μPAD with a mechanical valve when adding different volumes of the red solution. FIGS. 5D1, 5D2, 5D3, 5D4, and 5D5 show time-lapsed images of loading solutions at the beginning and finishing the two steps automatically on a μPAD with the mechanical valve.

FIGS. 6A1, 6A2, 6A3, 6A4, 6A5 and 6A6 illustrate time-lapsed images of the addition of solutions at the beginning and end of an automated four-step μPAD with a horizontal mechanical valve.

FIGS. 7A1, 7A2, 7A3, 7A4, and 7A5 illustrate time-lapse images of fluid flow in a μPAD with a localized dissolvable delay and a mechanical valve. FIG. 7B shows surface-enhanced Raman scattering (SERS) response of the μPAD to different concentrations of cardiac troponin I (cTnI) in a phosphate-buffered saline (PBS) solution. The inset is the SERS spectra for 0 ng/mL to 0.5 ng/mL of cTnI. The peak intensity at around 1614 cm⁻¹ (C—C and N-phenyl ring stretches in malachite green isothiocyanate) is used. The SERS intensity value in the response curve represents the summed value of the peak intensities in a 2.9 mm×0.9 mm area on the test line. Each concentration was tested using three replicates.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

A microfluidic paper-based analytical device (μPAD) is a platform composed of hydrophilic and hydrophobic channel networks in paper. Paper has the merits of abundance, low-cost, ease of disposability, ease of manipulation, being environmentally friendly, and having compatibility with biological sample fluids. Due to these advantages, paper-based devices have demonstrated their capability for being used in implementing assays, especially in point-of-care testing (POCT). However, most of these paper-based devices perform assays without controlling the fluid. Fluidic control in a μPAD is important in implementing high-performance assays. For instance, controlling the fluid to flow at a slower flow rate in paper-based devices can improve the sensitivity of the assay. A fluidic control valve can also be used to implement multi-step assays, such as those that include sequential loading, incubation, and washing. Thus, μPADs capable of implementing a multi-step protocol would help to translate high-performance assays into point-of-care (POC) settings.

As such, developing μPADs with fluidic control is important to implement multi-step assays and provide high sensitivities. However, current localized delays in μPADs, for example, those made of sucrose, have a limited ability to decrease the flow rate. In addition, existing μPADs for automatic multi-step assays are limited by their need for auxiliary instruments, their false activation, or their unavoidable tradeoff between available fluid volumes and temporal differences between steps.

In sum, a need exists for more effective microfluidic devices and methods for fluid flow. Various embodiments of the present disclosure address the aforementioned need.

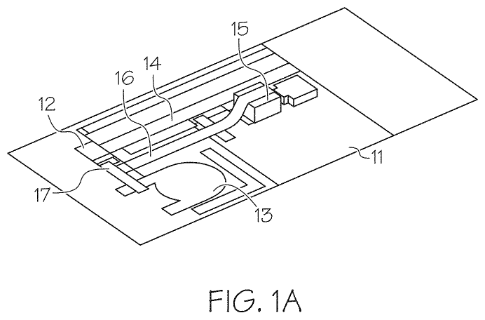

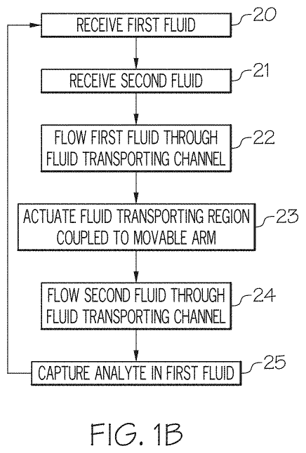

In some embodiment, illustrated in FIG. 1A, the present disclosure pertains to a microfluidic device (10) that includes a substrate (11) having an inlet region (12), a fluid storage region (13), and a fluid transporting channel (14) in fluid communication with the inlet region (12). In some embodiments, the fluid transporting channel (14) is a hydrophilic channel.

As further illustrated in FIG. 1A, the microfluidic device (10) further includes an expandable component (15) in fluid communication with the fluid transporting channel (14) and coupled to a movable arm (16), and a fluid transporting region (17) coupled to the movable arm (16) and operable to be moved in a horizontal direction parallel to the fluid transporting channel (14) to thereby form fluidic contact between the inlet region (12) and the fluid storage region (13) upon expansion of the expandable component (15).

Additional embodiments of the present disclosure pertain to fluid flow, such as through the utilization of the microfluidic devices of the present disclosure (e.g., microfluidic device 10, as illustrated in FIG. 1A). In some embodiments illustrated in FIG. 1B, the methods of the present disclosure include one or more of the following steps: of receiving a first fluid at an inlet region on a substrate (step 20) (e.g., inlet region 12 on substrate 11, as illustrated in FIG. 1A); receiving a second fluid at a fluid storage region on the substrate (step 21) (e.g., fluid storage region 13 on substrate 11, as illustrated in FIG. 1A); flowing the first fluid through a fluid transporting channel on the substrate in fluid communication with the inlet region (step 22) (e.g., fluid transporting channel 14 in fluid communication with inlet region 12, as illustrated in FIG. 1A); actuating a fluid transporting region that is coupled to a movable arm operable to be moved in a horizontal direction parallel to the fluid transporting channel via expansion of an expandable component in fluid communication with the fluid transporting channel (step 23) (e.g., actuation of fluid transporting region 17, which is coupled to movable arm 16 and operable to be moved in a horizontal direction parallel to fluid transporting channel 14 via expansion of expandable component 15, which is in fluid communication with fluid transporting channel 14, as illustrated in FIG. 1A); flowing the second fluid through the fluid transporting channel (step 24) (e.g., flowing the second fluid from fluid storage region 13 through fluid transporting channel 14 via fluid transporting region 17 and inlet 12, as illustrated in FIG. 1A), and capturing an analyte in the first fluid (step 25). In some embodiments, the method can be repeated.

As set forth in more detail herein, the microfluidic devices and methods of fluid flow of the present disclosure can have numerous embodiments. For instance, the microfluidic devices of the present disclosure can include various substrates having different fluid regions and fluid transporting channels. Furthermore, the microfluidic device can include expandable components coupled to a movable arm to provide for automatic fluid flow through the microfluidic devices.

Additionally, the microfluidic devices of the present disclosure can have various flow delaying mechanisms. Furthermore, the microfluidic devices of the present disclosure can be utilized in fluid flow. In some embodiments, fluid flow can be performed automatically without the use of external pumps and electronic or other auxiliary components.

Microfluidic Devices

As set forth in more detail herein, the microfluidic devices of the present disclosure can include various substrates having different regions and fluid transporting channels. In some embodiments, the different regions are operable to become in fluid communication with one another. Additionally, the microfluidic devices of the present disclosure can include various types of delays to control flow rate through the fluid transporting channel.

Substrates

As outlined in further detail herein, the microfluidic devices of the present disclosure can utilize various substrates. For example, in some embodiments, the substrate can include, without limitation, paper, cellulose paper, chromatography paper, filter paper, Whatman Grade 1 chromatography paper, Whatman Grade 1 filter paper, Whatman Grade 2 filter paper, Whatman Grade 3 filter paper, Whatman Grade 4 filter paper, Whatman Grade 591 filter paper, Whatman Grade 595 filter paper, Whatman Grade 598 filter paper, Fisherbrand quantitative grade filter paper, Fisherbrand qualitative grade filter paper, nitrocellulose paper, a membrane, Amersham protran nitrocellulose membrane, Whatman fast flow high performance nitrocellulose membrane, immunopore nitrocellulose membrane, and combinations thereof.

Fluid Regions/Fluid Transporting Channel

As set forth in further detail herein, the microfluidic devices of the present disclosure can include different regions. For example, in some embodiments, the microfluidic devices of the present disclosure can include an inlet region, a fluid storage region, and a fluid transporting region. In some embodiments, the inlet region is a sample inlet region. In some embodiments, the sample inlet region receives a sample (e.g., a biological sample). In some embodiments, the inlet region includes an analyte binding agent. In some embodiments, the fluid storage region is a buffer storage region. In some embodiments, the buffer storage region receives a buffer (e.g., a washing buffer).

Moreover, in some embodiments, the microfluidic device includes a fluid transporting channel in fluid communication with the inlet region. In some embodiments, the fluid transporting channel is a hydrophilic channel. In some embodiments, the microfluidic device includes an expandable component in fluid communication with the fluid transporting channel and coupled to a movable arm. In some embodiments, the expandable component can include, without limitation, a porous material, a material capable of absorbing a fluid, and combinations thereof. In some embodiments, the expandable component is a sponge.

In some embodiments, the fluid transporting region is coupled to the movable arm and operable to be moved in a horizontal direction parallel to the fluid transporting channel to thereby form fluidic contact between the inlet region and the fluid storage region upon expansion of the expandable component. In some embodiments, the expandable component expands after exposure to a first fluid. Moreover, in some embodiments, the substrate can include a control line in the fluid transporting channel. In some embodiments, the substrate includes a test line in the fluid transporting channel.

In some embodiments, the fluid transporting channel includes an analyte binding agent. In some embodiments, the inlet region includes an analyte binding agent. In some embodiments, the analyte binding agent is a functionalized analyte binding agent. In some embodiments, the functionalized analyte binding agent is a particle that transduces with surface-enhanced Raman scattering (SERS)-active, fluorescent, absorptive, colorimetric, chemiluminescence, magnetic intensity, or combinations thereof. In some embodiments, the SERS-active particle is used to target cardiac troponin I (cTnI) or any other biomarker in blood, urine, saliva, sweat, tear, and combinations thereof in the first fluid.

In some embodiments, the fluid transporting channel includes a first analyte binding agent and the inlet region includes a second analyte binding agent. In some embodiments, the fluid transporting channel includes a DNA sequence, aptamer, antibody or any combination thereof for capturing the second analyte binding agent.

Multiple Fluid Storage Regions

As set forth in further detail herein, the microfluidic devices of the present disclosure can include multiple fluid storage regions. For example, in some embodiments, the substrate further includes a second storage region and a third storage region. In some embodiments, the fluid transporting region is operable to be moved in the horizontal direction parallel to the fluid transporting channel to thereby form fluidic contact between the inlet region and the second storage region. In some embodiments, the fluid transporting region is operable to be moved in the horizontal direction to the fluid transporting channel to thereby form fluidic contact between the inlet region and the third storage region. In some embodiments, the second storage region can include chemical reagents. In some embodiments, the third storage region can include chemical reagents. In some embodiments, each of the second and third storage regions can include chemical reagents. In some embodiments, the chemical reagents can include analyte binding agents.

In some embodiments, the substrate further includes one or more additional fluid storage regions. In some embodiments the fluid transporting region is operable to be moved in the horizontal direction to the fluid transporting channel to thereby form fluidic contact between the inlet region and each of the one or more additional fluid storage regions. In some embodiments, one or more of the one or more additional fluid storage regions can include chemical reagents. In some embodiments, each of the one or more additional fluid storage regions can include chemical reagents. In some embodiments, the chemical reagents can include analyte binding agents.

Flow Rate Delays

In some embodiments, the microfluidic devices of the present disclosure can include various delays to control flow rate through the fluid transporting channel. For example, in some embodiments, the microfluidic devices of the present disclosure further include a localized dissolvable delay in contact with the fluid transporting channel to control flow rate of a first fluid through the fluid transporting channel. In some embodiments, the localized dissolvable delay is a gate.

In some embodiments, the localized dissolvable delay is a region composed of a mixture that can include, without limitation, sugar-based compositions, sucrose compositions, fructose compositions, sucrose and fructose compositions, trehalose compositions, glucose compositions, glucose and sucrose compositions, galactose compositions, dextran compositions, isomalt compositions, maltitol compositions, lactitol compositions, soluble macromolecules, water-soluble polymers, polyvinyl alcohol, polyvinyl alcohol compositions, pullulan, pullulan composites, glycerol, polysorbate 20, and combinations thereof.

In some embodiments, delay is modulated via a mechanism that can include, without limitation, molecular weight of constituents in the mixture, concentration of the mixture, constituents in the mixture, and combinations thereof. In some embodiments, the delay region is deposited on the fluid transporting channel. In some embodiments, the delay region is painted on the fluid transporting channel.

Method of Fluid flow

As set forth in further detail herein, the microfluidic devices of the present disclosure can be utilized for various purposes. For example, in some embodiments, the microfluidic devices of the present disclosure can be utilized for fluid flow. In general, the method for fluid flow includes one or more of the following steps of: (1) receiving a first fluid at an inlet region on a substrate; (2) receiving a second fluid at a fluid storage region on the substrate; (3) flowing the first fluid through a fluid transporting channel on the substrate in fluid communication with the inlet region; (4) actuating a fluid transporting region coupled to a movable arm operable to be moved in a horizontal direction to the fluid transporting channel via expansion of an expandable component in fluid communication with the fluid transporting channel; and (5) flowing the second fluid through the fluid transporting channel.

In some embodiments, the methods of the preset disclosure can be performed automatically. In some embodiments, the methods of the present disclosure can be performed without the use of an external pump to flow the first fluid or the second fluid through the microfluidic device. In some embodiments, the microfluidic devices and the methods of use thereof provide for automatic dispensing of one or more fluids through the microfluidic device.

First Fluids

As set forth in further detail herein, the methods of the present disclosure can utilize various first fluids. For example, in some embodiments, the first fluid is a biological sample fluid. In some embodiments, the biological sample fluid can include, without limitation, blood, urine, saliva, sweat, a tear, and combinations thereof. In some embodiments, the first fluid can include a component necessary for point-of-care testing.

In some embodiments, the first fluid can include various analyte binding agents. In some embodiments, the first fluid can include one or more analytes that react and/or bind with an analyte binding agent within the microfluidic device. In some embodiments, the analyte binding agent is an antibody. In some embodiments, the analyte binding agent is an aptamer, antibody, DNA strand or combinations thereof.

In some embodiments, the first fluid includes components to help with the diagnosis and early treatment of myocardial infarction. In some embodiments, the first fluid can include cTnI or other biomarker in blood, urine, saliva, sweat, a tear, and combinations thereof.

Second Fluids

As set forth in further detail herein, the methods of the present disclosure can utilize various second fluids. For example, in some embodiments, the second fluid can include a buffer solution. In some embodiments, the buffer solution is a washing solution. In some embodiments, the second fluid is phosphate-buffered saline (PBS).

Flow Rate Delays

As set forth in further detail herein, the methods of the present disclosure can further include a step of delaying flow rate of the first fluid through the fluid transporting channel. For example, in some embodiments, the delaying of the first fluid through the fluid transporting channel is conducted via a delay region. In some embodiments, the delay region is a gate.

In some embodiments, the delay region is composed of a mixture, that can include, for example, sugar-based compositions, sucrose compositions, fructose compositions, sucrose and fructose compositions, trehalose compositions, glucose compositions, glucose and sucrose compositions, galactose compositions, dextran compositions, isomalt compositions, maltitol compositions, lactitol compositions, soluble macromolecules, polymers, polyvinyl alcohol, polyvinyl alcohol compositions, water-soluble polymers, pullulan, pullulan composites, glycerol, polysorbate 20, and combinations thereof.

In some embodiments, the delaying flow of the first fluid is modulated via a mechanism that can include, without limitation, molecular weight of constituents in the mixture, concentration of the mixture, constituents in the mixture, and combinations thereof.

Analyte Capture

As set forth in further detail herein, the methods of the present disclosure can additionally include various steps for analyte capture. For example, in some embodiments, the fluid transporting channel includes a first analyte binding agent and the inlet region includes a second analyte binding agent. In such embodiments, the methods of the present disclosure can further include one or more of the following steps of: (1) resuspending the second analyte binding agent with the first fluid; (2) capturing an analyte in the first fluid with the second analyte binding agent; and (3) capturing the second analyte binding agent and the analyte with the first analyte binding agent.

Furthermore, in some embodiments, the fluid transporting channel includes a component capable of binding to the second analyte binding agent. In some embodiments, the fluid transporting channel includes a DNA strand, aptamer, antibody, or combinations thereof. In such embodiments, the method can include capturing the second analyte binding agent with the DNA strand, aptamer, antibody, or combinations thereof.

In some embodiments, the methods of the present disclosure provide for non-specific binding at reaction sites of the microfluidic device. In some embodiments, the reaction sites can include, for example, one or more sites in the fluid transporting channel.

Removal of Uncaptured Components

As set forth in further detail herein, the methods of the present disclosure can additionally include various steps for removal of uncaptured components. For example, in some embodiments, the methods of the present disclosure can further include one or more of the following steps of: (1) washing the inlet region and the fluid transporting channel with the second fluid; and (2) removing uncaptured components in the first fluid. In some embodiments, the components can include, without limitation, particles, molecular dyes, enzymes, and combinations thereof.

Sample Fluid Testing

As set forth in further detail herein, the methods of the present disclosure can additionally include various types of sample fluid testing. For example, in some embodiments, the methods of the present disclosure can further include reading a signal from the fluid transporting channel. In some embodiments, the reading is conducted via surface enhanced Raman spectroscopy, colorimetry, absorbance, fluorescence, chemiluminescence, magnetic intensity, and combinations thereof. In some embodiments, analytes captured via the methods of the present disclosure can be tested. In some embodiments, analytes captured via the methods of the present disclosure can be detected.

In some embodiments, the testing includes point-of-care testing. In some embodiments, the point-of-care testing is detection and/or monitoring of a biomarker for diagnosis or treatment of a disease such as, for example, myocardial infarction.

Applications and Advantages

The present disclosure can have various advantages. For instance, in some embodiments, the microfluidic devices of the present disclosure have at least the following valuable features: (1) the microfluidic devices eliminate the dependence on auxiliary instruments for automatic multi-step processes; (2) with no auxiliary instruments, multi-step processes can be achieved by the expandable component actuated by the fluids; (3) controlling the fluid flow in different channels such that the fluid arrives at the reaction point at different times in the microfluidic devices; (4) the microfluidic devices are cost effective and easy to use; (5) the microfluidic devices have increased accuracy over currently available devices for fluid flow; (6) the microfluidic devices prevent false actuations occurred in devices using vertical movements; (7) the delay region is in a localized region in the fluid transporting channel and leaves more vacant region for other components; (8) the delay region is simple to make; (9) the delay region increases flow time efficiently; and (10) the microfluidic devices provide for analyte binding at reaction sites within the microfluidic devices. Furthermore, the microfluidic devices and methods of fluid flow of the present disclosure provide multi-step assays with high sensitivities.

As such, the microfluidic devices of the present disclosure can be utilized in various manners and for various purposes. For instance, in some embodiments, the microfluidic devices of the present disclosure can be utilized for automated fluid flow and/or point-of-care testing.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Paper Microfluidic Device with a Horizontal Motion Valve and a Localized Delay for Automatic Control of a Multi-Step Assay

A microfluidic paper-based analytical device (μPAD) is a cost-effective platform to implement assays, especially for point-of-care testing (POCT). Developing μPADs with fluidic control is important to implement multi-step assays and provide high sensitivities. However, current localized delays in μPADs made of sucrose have a limited ability to decrease the flow rate. In addition, existing μPADs for automatic multi-step assays are limited by their need for auxiliary instruments, their false activation, or their unavoidable tradeoff between available fluid volumes and temporal differences between steps. In this Example, a novel μPAD composed of a localized dissolvable delay and a horizontal motion mechanical valve for use as an automatic multi-step assay is demonstrated. A mixture of fructose and sucrose was used in the localized dissolvable delay and it provided an effective decrease in flow rate to ensure adequate sensitivity in an assay. The dissolvable delay effectively doubled the flow time. A mechanical valve using horizontal movement was developed to automatically implement a multi-step process. Two-step and four-step processes were enabled with the μPAD. Cardiac troponin I (cTnI), a gold standard biomarker for myocardial infarction, was used as a model analyte to show the performance of the developed μPAD in an assay. The designed μPAD, with the simple-to-make localized dissolvable delay and the robust mechanical valve, provides the potential to automatically implement high-performance multi-step assays toward a versatile platform for point-of-care diagnostics.

### Example 1.1. Introduction

A microfluidic paper-based analytical device (μPAD) is a platform composed of hydrophilic and hydrophobic channel networks in paper. Paper has the merits of abundance, low-cost, ease of disposability, ease of manipulation, being environmentally friendly, and having compatibility with biological samples. Due to these advantages, paper-based devices have demonstrated their capability for being used in implementing assays, especially in point-of-care testing (POCT). However, most of these paper-based devices perform assays without controlling the fluid. Fluidic control in a μPAD is important in implementing high-performance assays. For instance, controlling the fluid to flow at a slower flow rate in paper-based devices can improve the sensitivity of the assay. A fluidic control valve can also be used to implement multi-step assays, such as those that include sequential loading, incubation, and washing. Thus, μPADs capable of implementing a multi-step protocol would help to translate high-performance assays into point-of-care (POC) settings.

Efforts to control flow rate have included several designs such as changing the geometries of the channel or altering the permeability of the paper by using materials including wax, agarose, sucrose, and trehalose. Sucrose is a popular permeability altering material since it is a water-dissolvable, abundant and low-cost material. It is also currently used in the preservation of reagents in many paper-based analytical devices and has minimal effect on many assay chemistries. A wide range of time delays have been achieved by using a sucrose-based dissolvable delay. However, the dissolvable delay using sucrose typically covers the whole channel and the method used makes it difficult to quantify the sucrose applied on the channel. This type of dissolvable delay is not suitable for the localization of sucrose in a specific position in the channel. In one study, a sucrose-based dissolvable barrier was fabricated by depositing a sucrose solution on paper using a modified craft-cutting instrument. Using this method, the position and volume of the localized sugar delay are well controlled. However, 5 drawings on a Whatman grade 1 chromatography paper only provided a delay time of around 48 s, which might be enough for some applications, but may be too short for POC applications that often require more time. Therefore, a simple one-step method to precisely make a localized dissolvable sugar delay, which efficiently increases the flow time and decreases the flow rate would be desirable.

To achieve multi-step processes using μPADs, various methods have been developed. Automatic systems are easier to use especially for untrained users. To retain the cost-effectiveness of μPADs, eliminating the dependence on auxiliary instruments for automatic multi-step processes is desired. With no auxiliary instruments, multi-step processes can be achieved by controlling the fluid flow in different channels such that the fluid arrives at the reaction point at different times. Perturbation of fluid flow speeds in μPADs may occur using the following techniques: channel geometry variations, dissolvable materials, variation in paper wettability, and carving channels on paper. Limitations in these methods include loss of effective fluid volume, which can affect the assay reaction and fluid mixing with the dissolvable materials leading to a change in assay chemistries. Designs using folded paper actuators and compressed cellulose sponges have the potential to overcome these limitations as each method utilizes valve actuation for multi-step processes. In the two designs, the connection or separation of channels for fluid of different steps are controlled by vertical movements generated by a sponge or a folded paper actuator. However, the channels that are designed to stay spatially separated at different heights have the potential to become falsely connected before actuation. To ensure its performance, extra care is needed to prevent false actuations between channels. Developing a μPAD that uses horizontal movement of the actuator could largely avoid this problem.

In this Example, a simple and robust system that includes a mechanical valve using horizontal movement to implement a multi-step process in a μPAD along with a localized dissolvable delay to control flow rate and enhance assay sensitivity is described. This Example was the first demonstration of using a mixture of fructose and sucrose in a dissolvable delay. The effect of the ratio of fructose to sucrose, the volume of the mixture, and the position of the dissolvable delay were analyzed. A one-step method using pipetting and wax-printed scale lines was used to make the dissolvable delay in a localized region. In addition, a horizontal motion mechanical valve, a paper arm and a compressed sponge was used to achieve a multi-step process. The paper arm uniquely transformed the vertical movement of the compressed sponge into a horizontal movement. The developed μPAD, that included the localized dissolvable delay and the mechanical valve, was initially characterized with dye solutions and then tested on a model assay namely; a surface-enhanced Raman scattering (SERS) assay developed to target cardiac troponin I (cTnI), a clinically validated biomarker for myocardial infarction.

### Example 1.2. Materials

Whatman Grade 1 chromatography paper was purchased from GE Healthcare (IL, USA). Glass fiberpad (GFCP000800) was from EMD Millipore (MA, USA). Thick blot filter paper (#1703932) was purchased from Bio-Rad (CA, USA). The compressed rectangular sponge (43CC) was purchased from Sponge Producers Company (MO, USA). The mounting adhesive sheets were purchased from Michaels (TX, USA). Transparent sealing film (UC-500) was from Axygen (CA, USA). Food dye (red) were purchased from a local supermarket (TX, USA). Sucrose, D-(−)-fructose, sodium citrate tribasic dihydrate, gold(III) chloride trihydrate, (3-mercaptopropyl)trimethoxysilane (MPTMS), 2-propanol, tetraethyl orthosilicate (TEOS), ammonium hydroxide (28%), ethanol, sodium cyanoborohydride (NaBH₃CN), and sodium periodate (NaIO₄) were purchased from Sigma Aldrich (MO, USA). Malachite green isothiocyanate (MGITC) and green fluorescent particles (GO100) were purchased from Thermo Fisher Scientific (MA, USA). Carboxy-poly(ethylene glycol)-thiol (SH-PEG-COOH, M, 10 kDa) was purchased from Nanocs (NY, USA). N-hydroxysulfosuccinimide sodium salt (Sulfo-NHS) and N-ethyl-N′-(3-dimethylaminopropyl)carbodiimide hydrochloride (EDC-HCl) were from CovaChem (IL, USA). (3-triethoxysilyl)propylsuccinic anhydride (TEPSA) was purchased from Gelest (PA, USA). Cardiac Troponin I (cTnI) was purchased from GenScript (NJ, USA). The sequence of aptamers for cTnI assay was reported previously. In this Example, aptamer 1 (5′-amine-2 hexa-ethyleneglycol spacers-CGTGC AGTAC GCCAA CCTTT CTCAT GCGCT GCCCC TCTTA-3′) (SEQ ID NO: 01) and aptamer 2 (5′-amine-2 hexa-ethyleneglycol spacers-CGCAT GCCAA ACGTT GCCTC ATAGT TCCCT CCCCG TGTCC-3′) (SEQ ID NO: 02) were used. Both aptamers and the control line DNA strand (5′-amine-hexa-ethyleneglycol spacer-GGACA CGGGG AGGGA ACTAT GAGGC AACGT TTGGC ATGCG-3′) (SEQ ID NO: 03) were from Integrated DNA Technologies (IA, USA). Milli-Q ultrapure water (18.2 MΩ cm⁻¹) was used in all the procedures.

### Example 1.3. Instrumentation

The wax was printed on the paper using a ColorQube 8570 wax printer (Xerox, USA). The fluid flow on the paper was recorded using a webcam (Logitech Webcam c922). The fluorescent images and videos were recorded using a benchtop Nikon Eclipse Ti2-U fluorescence microscope (Nikon, Japan). Scanning electron microscope (SEM) images were acquired on a JEOL JSM-7500F (JEOL, Japan). Transmission electron microscopy (TEM) images were acquired on a JEOL JEM-2010 (JEOL, Japan). All SERS spectra were collected using a Thermo Scientific DXR Raman confocal microscope with a 780 nm laser. The magnification and numerical aperture of the objective were 10× and 0.25, respectively. The spectral range was from 200 cm⁻¹ to 1800 cm⁻¹, and the spectral resolution was 3.0-4.1 cm⁻¹. Samples were excited with a 24 mW laser using 2-sec exposure per reading. All spectra were baseline corrected.

### Example 1.4. Preparation of Paper Substrates

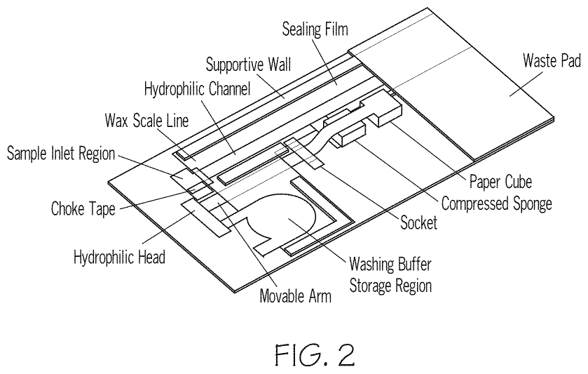

Hydrophobic wax barriers and wax scale lines were patterned on Whatman grade 1 chromatography paper. The paper with wax patterns was then heated in an oven at 120° C. for 2 min to melt the wax and form the hydrophobic boundary. An adhesive backing sheet was taped on the bottom of the paper. Two pieces of hydrophobic tape, labeled as “choke tape” in FIG. 2, were used to form a sample inlet region at the beginning of the hydrophilic channel. The choke tape was used to direct fluid samples into the porous paper matrix and to prevent them from flowing onto the surface of the paper.

### Example 1.5. Horizontal Motion Mechanical Valve

FIG. 2 shows the design of the lateral motion mechanical valve in the μPAD. A compressed sponge (6 mm×6 mm×2.5 mm) was fixed to the paper using adhesive tapes. The overlapping region between the sponge and the hydrophilic channel was 6 mm×3 mm. The movable arm is composed of a hydrophobic wax body and a hydrophilic head. A socket to guide the horizontal movement of the arm was made using paper and adhesive tape. The arm went through the socket, and the end of the hydrophobic body was taped on a paper cube with a height of 2.5 mm. The paper cube, which was taped on the wax region, was used to keep the end of the arm high and reduce the downward pressure applied on the compressed sponge. A transparent sealing film was covered on top of the hydrophilic region with a 0.5 mm gap between the sealing film and the paper. Adhesive sheets with a thickness of 0.5 mm were used as a supportive wall to form the gap. The fabricated paper was then stored in a sealed container before use.

Performance of the mechanical valve was characterized using red and blue food colorings, diluted in phosphate-buffered saline (PBS). 150 μL of the blue solution was loaded in the washing solution storage area, followed by loading 75 μL of the red fluid into the inlet of the μPADs. Movement of the two fluids was recorded using a webcam. Height change of the compressed sponge was measured using a digital caliper.

### Example 1.6. Localized Dissolvable Delay

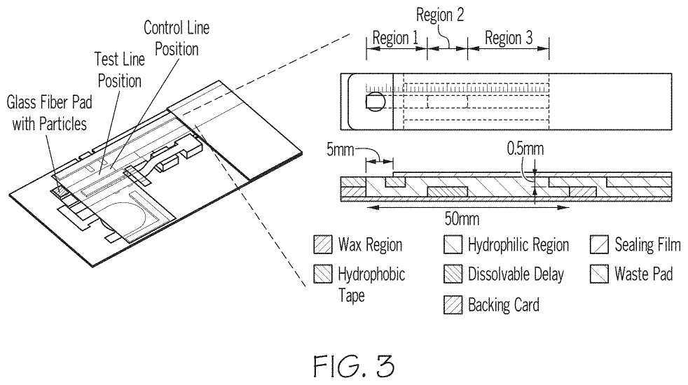

FIG. 3 shows the overall design of the μPAD with mechanical valve and the localized dissolvable delay for a multi-step assay. The dissolvable delay portion was initially fabricated and tested before adding the mechanical valve. To make the dissolvable delay region, the following process was used. Sucrose was mixed with water at room temperature for 2 days to yield a saturated sucrose solution (concentration of ˜2 g/mL at 20° C.). Concentrations of 0.2 g/mL, 0.6 g/mL, 1 g/mL, 1.4 g/mL, and 1.8 g/mL of sucrose solutions were made by adding different volumes of water into the saturated sucrose solution. To make sucrose/fructose mixtures, 0.5 g, 1 g, 1.5 g, and 2 g fructose was added into 1 mL of the sucrose solutions to get sucrose solutions with 0.5 g/mL, 1.0 g/mL, 1.5 g/mL, and 2.0 g/mL fructose, respectively. Solutions were agitated for 2 hours to aid dissolution.

The mixture was deposited on the hydrophilic channel of the paper using reverse pipetting, meanwhile, the wax scale lines were used to indicate the position of the drop. The paper containing the sucrose/fructose mixture solution was left at room temperature with 55% humidity until the solution is fully dispersed across the paper. The paper was then dried in an oven at 45° C. for 12 hours. After drying the mixture, a dissolvable delay was formed in a small localized area. The hydrophilic region was then covered with a sealing film using adhesive sheets (thickness of 0.5 mm) as a supportive wall. The μPAD was then stored in a sealed container with desiccant before use.

The dissolvable delay was characterized using a webcam to record movement of the fluid front in the hydrophilic channel after loading 50 μL of PBS containing red dye. Characterization of the particle count of 1.1 m green fluorescent particles in the hydrophilic channel with and without a localized dissolvable delay was measured using a fluorescent microscope. Specifically, movement of the fluorescent particles at 10 mm down the channel in the μPADs was recorded at 300 s, 600 s, 900 s, 1200 s, 1440 s, and 1680 s, after loading the fluorescent particles in 50 μL of water. The particle count was obtained using a Lagrangian particle tracking algorithm. One-way analysis of variance (ANOVA) in OriginLab was used to determine whether the flow times were significantly different when using different dissolvable delays.

### Example 1.7. Aptamer Based SERS Assay Using μPAD

As shown in FIG. 3, a test line and a control line were formed on the μPAD for the multi-step assay. To develop the test line and the control line, aptamer 1 and the control line DNA strand were immobilized on the hydrophilic channel of cellulose paper using a modified process based on the Schiff base plus reduction method. After immobilization of aptamer 1 on the μPAD, the localized dissolvable delay and the mechanical valve was fabricated using the processes mentioned in the previous section.

SERS-active silica shell particles functionalized with MGITC as the Raman reporter molecule were synthesized. Aptamer 2 was then attached to the synthesized silica shell particle using TEPSA as a linker. The synthesized aptamer functionalized SERS active particles were then dried on the glass fiber pad and cut into 4 mm×4 mm squares. The glass fiber pad with the particles was put at the inlet of the μPAD, as shown in FIG. 3.

To test the performance of the developed μPAD, a model assay for the detection of cTnI solutions was developed. Specifically different concentrations of cTnI (0, 0.01, 0.05, 0.1, 0.2, 0.5, 1 ng/mL) were prepared in PBS (PH 7.4). The assay was implemented by loading 150 μL of washing buffer (PBS with 0.25% Tween 20) in the washing solution storage area and loading 75 L of the cTnI solution in the sample inlet. After loading the solutions, the μPAD automatically completed the steps of wicking the sample to the reaction region and washing excess reagents. After the washing solution was completely wicked, the SERS signal of the test line in the μPAD was measured using a Raman microscope.

### Example 1.8. Measuring the Delay Time with the Localized Dissolvable Delay

The dissolvable delay portion of the μPAD (FIG. 3) was first tested before introducing the mechanical valve. The delay was positioned in the middle (Region 2) of a 50 mm hydrophilic channel. Region 1 and Region 3 represent the areas before and after the localized delay, respectively. Evaporation, which could cause loss of sample solution and change in flow rate, cannot be neglected in a long-time reaction. Thus, a sealing film was used to cover the hydrophilic region in the μPAD. The evaporation test showed a significant reduction in the percent of mass loss with time when the sealing film was used indicating reduced evaporation.

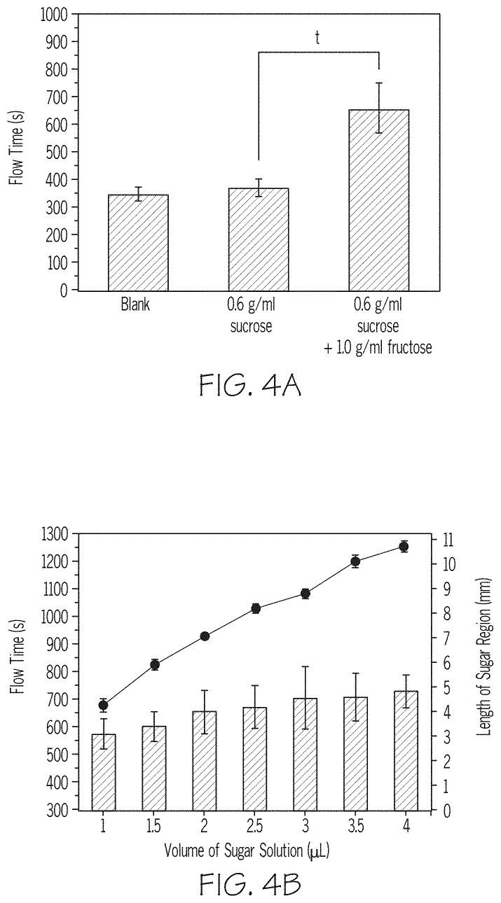

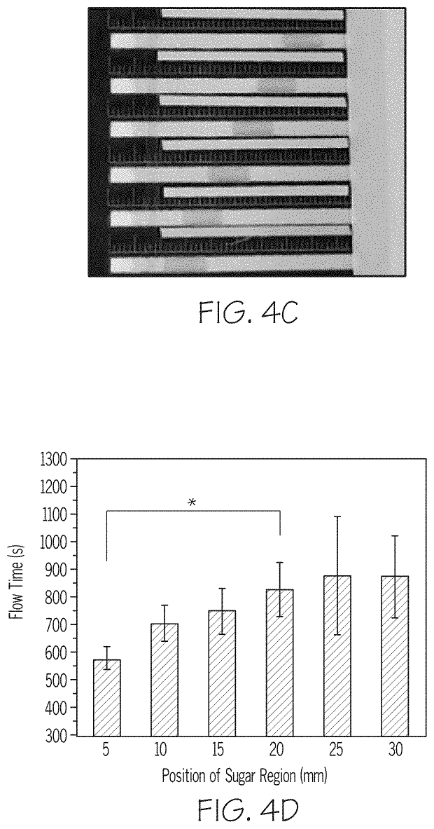

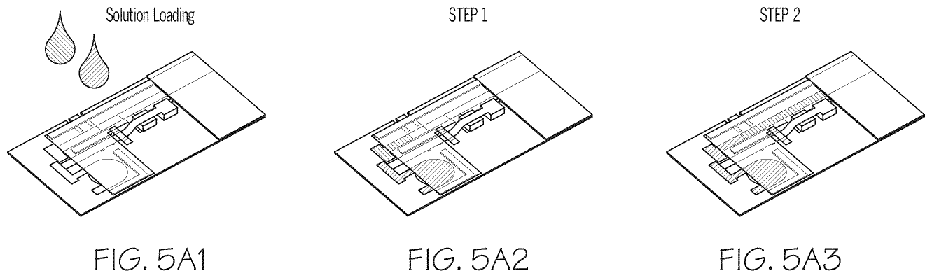

Fructose, a monosaccharide, that combines with glucose to form sucrose, has been used to slow down crystallization of sucrose because fructose attaches on the major growing face of a sugar crystal and inhibits the incorporation of the sucrose. Here, fructose and mixtures of fructose and sucrose were evaluated for their performance in providing a localized dissolvable delay. Flow delay was tested by measuring the time the fluid front took to reach the location in Region 3 (40 mm). Based on results obtained, 0.6 g/mL sucrose and 1.0 g/mL fructose were selected and combined to form the optimal sucrose/fructose mixture that provided a long delay time. Further, the flow time of the solution through the μPAD with a localized delay composed of the fructose/sucrose mixture was compared with a blank μPAD and one with localized delays made of just 0.6 g/mL sucrose. As shown in FIG. 4A, the localized sucrose/fructose delay provided a flow time of 661 s, close to 2 times the flow time in a μPAD without a dissolvable delay (348 s), and was better than only sucrose. The longer flow time of the sucrose/fructose mixture may be attributed to the higher-viscosity mixture dispersing slower across the paper, with the slower speed in forming sucrose crystals, and/or could be the better penetration in the paper pores of fructose. Due to these effects, less pores present after drying. More effectively filled pores possibly decreased the cylindrical pore radius, which increased flow time and reduced flow rate. Moreover, as the sugar was effectively dissolved, both fructose and sucrose increased fluid viscosity and the fluid was effectively slowed down due to the higher viscosity.

### Example 1.9. The Effect of Dissolvable Delay Region Width and Distance from the Inlet

In this Example, the dissolvable delay is in a localized region in the μPAD and this limited length of the sugar region enables a flexible position for the assay design in μPADs. The lengths of the sugar region were affected by the volume of the sucrose/fructose mixture dried on the channel. FIG. 4B shows that the larger the volume of sugar solution, the longer the length of the sugar region. Dissolvable delays with lengths ranging from 4.3 mm to 10.8 mm were achieved. FIG. 4B also shows that the longer length of the sugar region, the longer the flow time, but the flow time was effectively constant after the length of the sugar region reached 8.2 mm (i.e., 2.5 μL of sugar solution).

The increase in flow time with the increased volume of sugar solution could be attributed to a longer Region 2 with a small cylindrical pore size, and a higher fluid viscosity in both Region 2 and Region 3. Based on FIG. 4B, because the increase of flow time slowed down at 2.5 μL, 2.5 L of 0.6 g/mL sucrose with 1.0 g/mL fructose was selected due to its short length and long delay time.

The effect of the position of the sugar region was characterized, and FIG. 4C shows the μPAD with sucrose/fructose delays made at different positions down the channel and their different flow times. For a longer Region 1, the start of Region 2 that contains the sugar mixture is further away, which causes a slower dissolution. This slower dissolution allows the pore sizes to stay small and the paper to exhibit low porosity (i.e., the sugar mixture does not dissolve as quickly and so there is a smaller cylindrical pore radius) but also yields a lower viscosity (i.e., there is less material dissolved in the solution and traveling to Region 3). Notably, these two factors affect the flow time in opposite directions. From the result shown in FIG. 4D, the flow time was longer the farther the sugar region was from the inlet (5 mm to 20 mm). This suggests that the effect of the location on the sugar mixture region on keeping the cylindrical pore radius small was larger than the lower fluid viscosity. In addition, the FIG. showed that the flow time reached a plateau when the dissolvable sugar mixture region was further than 20 mm. At 15 mm an effective delay in flow was depicted with a flow time of 756 s, which was 2 times greater in the μPAD without a dissolvable delay. As a result, the 15 mm position was selected as the position for the localized dissolvable delay.

To use the μPAD for an assay, a test line with recognition elements was immobilized to capture target analytes and particles and a control line was created to capture excess particles and ensure the assay was working correctly. The test line and control line were designed to be located in Region 1 (before the delay) instead of Region 3 (after the delay) in order for the assay to avoid interaction with the dissolved sugar. Given the dissolved sugar mixture region starts at 15 mm from the inlet, the control line was located at 14 mm and the test line at 10 mm from the inlet. The flow at the 10 mm test line position was characterized using fluorescent particles. The localized delay decreased the particle flow at the 10 mm test line position. This result validated that the localized dissolvable delay effectively decreased flow rate and extended time for the assay to react.

### Example 1.10. Mechanical Valve Using a Two-Step Design

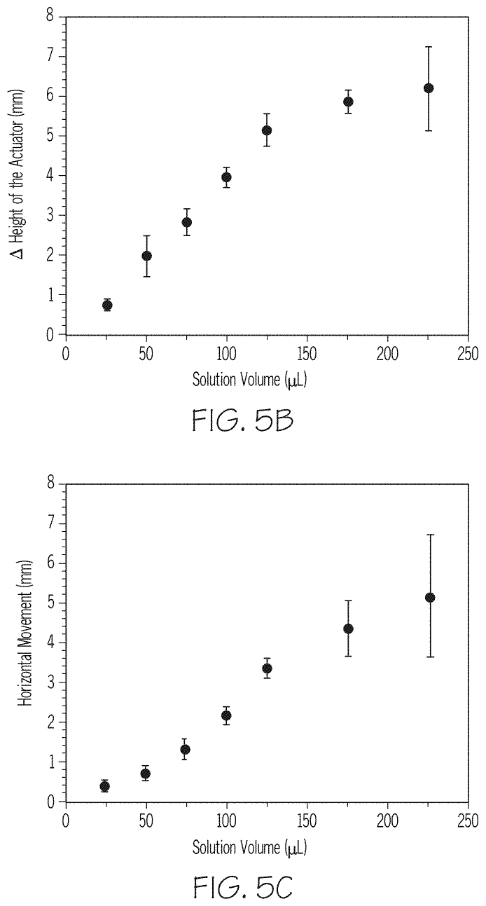

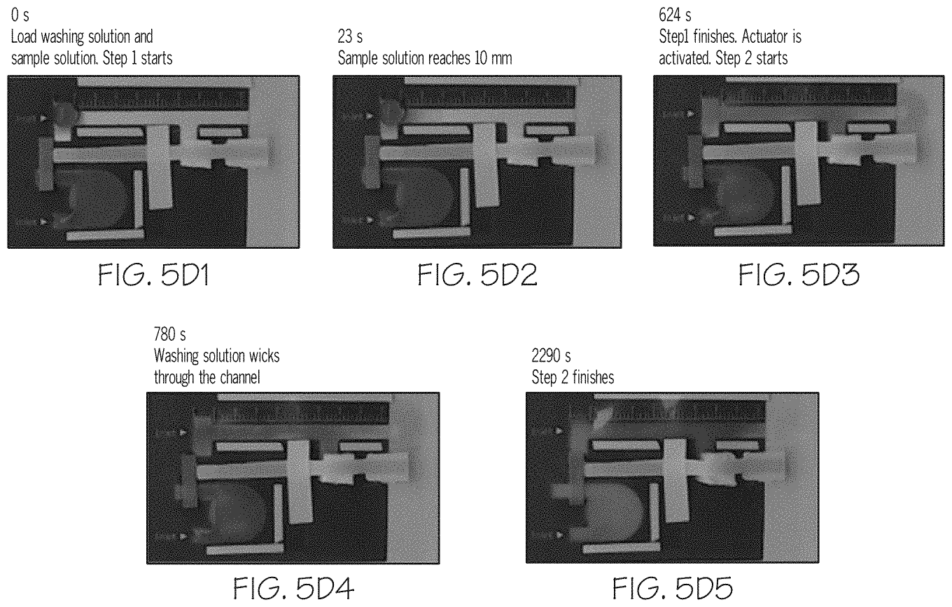

A novel mechanical valve using horizontal movement was developed using a paper arm and a compressed sponge. The two-step process is shown in FIGS. 5A1, 5A2, and 5A3. For Step 1, a red solution was loaded at the inlet and flowed down the hydrophilic channel. When the solution reached the compressed sponge, the sponge increased in height after it absorbed the sample solution. A paper arm and socket were used to transfer the vertical movement of the compressed sponge into a horizontal movement. When the paper arm moved, the hydrophilic head connected the washing buffer storage region with the hydrophilic channel, which then initiated Step 2, and the blue solution started to flow in the hydrophilic channel.

FIG. 5B shows the height change of the compressed sponge (actuator) related to the volume of the red solution loaded in the sample inlet. The larger the volume of the red solution, the larger the height change and, correspondingly, the larger the horizontal movement. FIG. 5B shows the height increased from 0.8 mm to 6.2 mm as the solution volume increased from 25 μL to 225 μL. However, the increase was much slower after the volume reached 125 μL and effectively stopped after the volume reached 175 μL. The reduced slope after 125 μL was primarily because the compressed sponge enlarged, became softer, and leaned to one side. Similarly, the horizontal movement of the hydrophilic head, depicted in FIG. 5C, showed a slowdown in the change after 125 μL. The slight leaning of the enlarged sponge also resulted in the moveable arm shifting to an angle, which contributed to the larger standard deviation in the horizontal movement. Thus, before initiating Step 2, the total volume of solution in Step 1 was kept smaller than 125 μL. Within this range, the horizontal movement allowed for a variation from 0.4 mm to 3.4 mm.

The time-lapsed images of the flow on the μPAD with the mechanical valve are shown in FIGS. 5D1, 5D2, 5D3, 5D4, and 5D5. At 0 s, the washing buffer storage region was loaded with 150 μL blue solution. The hydrophilic head was also wetted because wetting the hydrophilic head kept it in good contact with the paper surface and ensured a successful connection of the hydrophilic channel and the washing buffer storage region after actuation of the mechanical valve. 75 μL red solution was also loaded and began to travel down the channel reaching the mechanical valve sponge actuator after 624 s. The mechanical valve then automatically initiated Step 2, and the blue solution began loading and finished flowing at roughly 2290 s. It can be observed that the design provided a consistent control in the time to open the valve and initiate Step 2.

### Example 1.11. Mechanical Valve Using a Four-Step Design

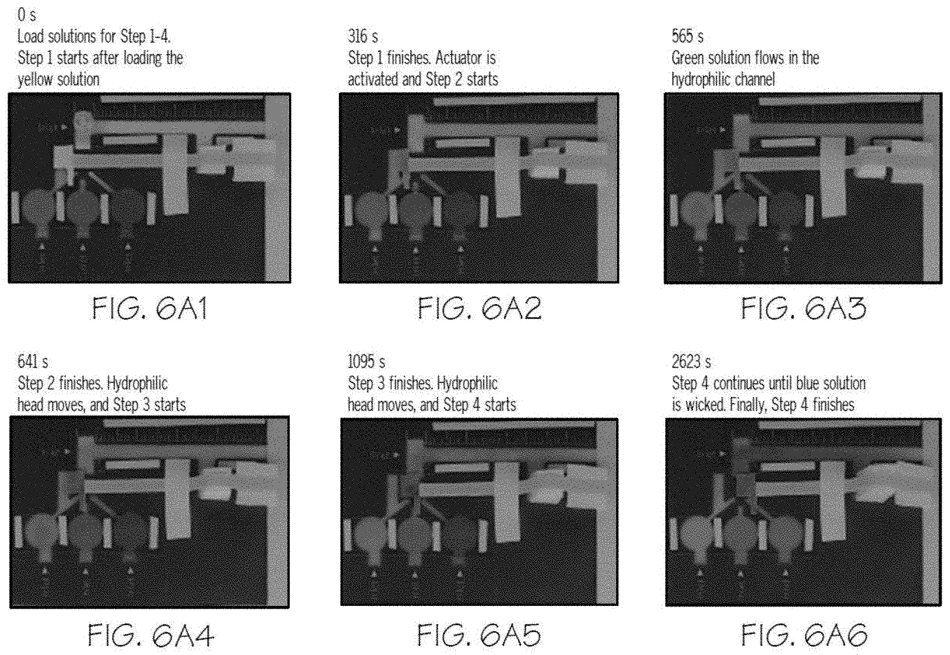

In addition to the two-step process, the mechanical valve was modified to achieve a four-step process as characterized in FIGS. 6A1, 6A2, 6A3, 6A4, 6A5 and 6A6. The modified μPAD was tested using PBS with four different colored solutions. Initially, 50 μL of the green, red, and blue solutions were loaded in the solution storage areas and then 50 μL of the yellow solution was loaded in the inlet of the μPAD. After loading the four solutions, the μPAD automatically finished the four-step process. As depicted in FIGS. 6A1, 6A2, 6A3, 6A4, 6A5 and 6A6, the yellow solution starts to flow in the channel after 0 s and is absorbed by the sponge. The valve is actuated and the green solution starts to flow at roughly 316 s, is absorbed by the sponge, causing the valve to open further and the red solution starts to flow at roughly 641 s. The red solution is absorbed by the sponge, opening the valve further allowing the blue solution to flow at roughly 1095 s. Compared to previous designs using a compressed sponge, this design uses horizontal movement instead of vertical movement to achieve the multi-step procedure. In this way, false actuations between the channels, that are designed to stay spatially separated at different heights before actuation, can be avoided.

### Example 1.12. Aptamer Based SERS Assay Design and Results Using the μPAD

A high sensitivity cTnI assay at the POC and outside the central lab, for example in an ambulance, is important to help the diagnosis and early treatment of myocardial infarction (MI). Toward that goal, a two-step aptamer-based SERS assay for cTnI was developed using the μPAD with a localized dissolvable delay and a mechanical valve. In the assay, aptamers were used as the recognition element and SERS-active silica shell particles were used to transduce the signal. Aptamer 1 was immobilized on the hydrophilic channel of the μPAD. A strong fluorescent intensity was observed on the test line region confirming the successful immobilization of aptamer 1 on the paper. Aptamer 2 functionalized SERS active particles were stored in the glass fiber inlet pad. To initiate the automatic two-step assay, the washing buffer and sample solution were loaded in the washing buffer storage region and sample inlet, respectively. After loading, the sample resuspended the aptamer 2 functionalized particles and they started to flow in the channel. When cTnI was present in a sample, it bound with aptamer 2 functionalized particles, which were then captured by aptamer 1 on the test line. The unbound aptamer 2 functionalized particles with no cTnI were captured on the control line. When the solution reached the delay region, the dissolvable delay slowed down the flow. After the sample solution dissolved the sugar, the fluid passed through and actuated the mechanical valve. The washing buffer was released and automatically flowed in the channel and washed off excess chemicals and unbound particles. The SERS signal of the test line was measured to determine the concentration of cTnI in the sample.

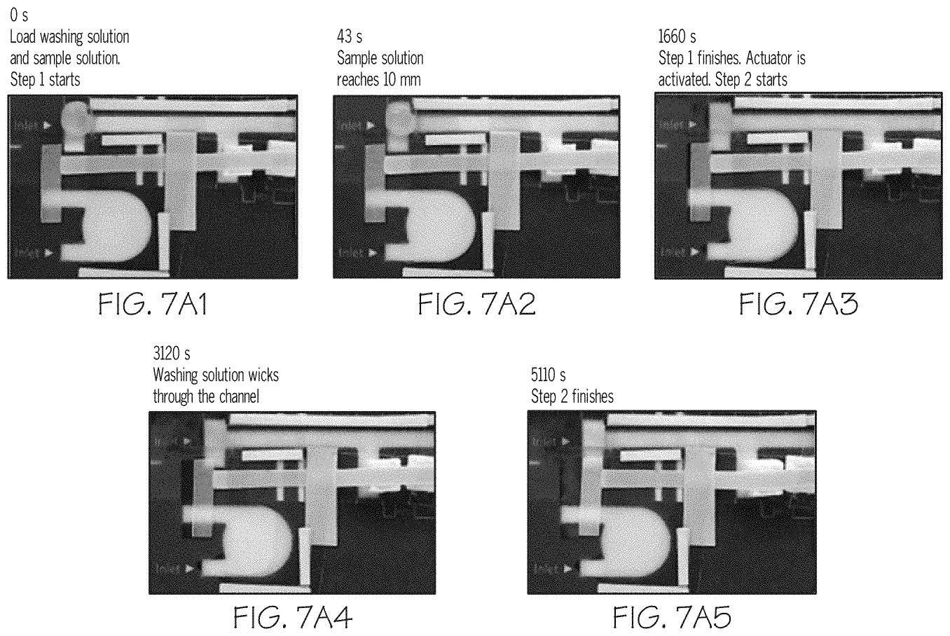

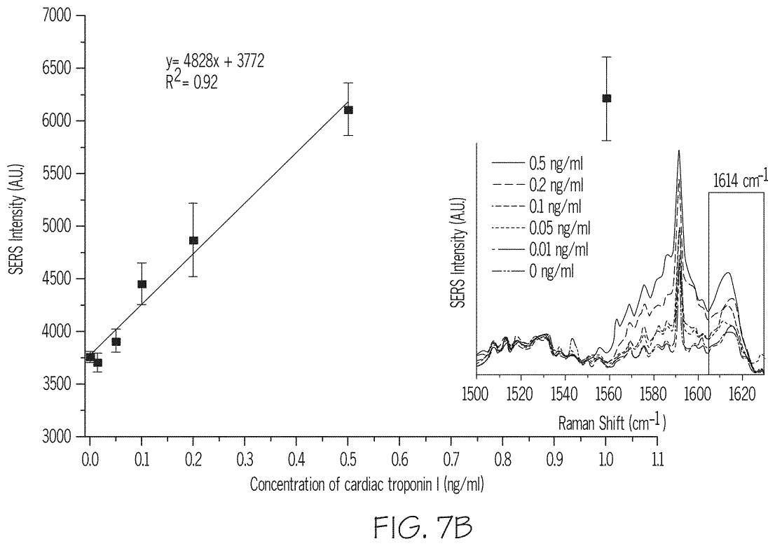

FIGS. 7A1, 7A2, 7A3, 7A4, and 7A5 show the time-lapse images of fluid flow in the μPAD. The contrast is lower here because nanoparticles with Raman reporters and a washing buffer were used instead of food dye solution. The SERS response to different concentrations of cTnI was acquired after the washing buffer finished flowing, and the results were shown in FIG. 7B. FIG. 7B shows the SERS intensity on the test line was linearly correlated with the concentration of the cTnI from 0 ng/mL to 0.5 ng/mL. The limit of detection (LOD) was 0.02 ng/mL. The focus for using this model assay was to show that the two-step SERS assay on the μPAD, with a localized dissolvable delay and a mechanical valve, could quantitatively determine the concentration of cTnI in a PBS buffer. Moreover, the assay response and the flow time in μPADs with and without the delay were compared. It was determined that, compared with the μPAD without the delay, the flow time in the μPAD with the dissolvable delay in step 1 was longer and the assay was more sensitive in the low concentration range, validating the slowing effect from the delay, which was designed to allow for more time for the assay components to react.

### Example 1.13. Conclusions

In this Example, a novel μPAD with a localized dissolvable delay and a horizontal motion mechanical valve was developed. A simple one-step method was used to fabricate the localized dissolvable delay region. A mixture of fructose and sucrose was shown to form an effective delay gate. Ratios of fructose to sucrose, volumes of the mixture, and positions of the delay were optimized. The localized dissolvable delay made of 2.5 μL of 0.6 g/mL sucrose with 1.0 g/mL fructose at 15 mm was determined to provide an effective increase of flow time to allow the assay to interact. A two-step and four-step process were successfully implemented using the horizontal mechanical valve with a paper arm and a compressed sponge. Because the paper arm in the mechanical valve moved in a horizontal direction, it prevented problems often encountered in vertical direction valves and provided robust activation. The developed μPAD was successfully used to automate an aptamer-based SERS assay for the quantitative detection of cTnI. In the assay, the localized dissolvable delay increased reaction time to ensure good detection sensitivity and the mechanical valve automatically actuated the washing step. The assay performance can be further improved if particles with stronger SERS signal are used and the μPAD production is automated. The developed μPAD was shown to provide a useful way to implement high-performance multi-step assays automatically and has the potential to play a role in converting current multi-step laboratory assays into simple point-of-care devices that have high performance yet remain easy to use.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A microfluidic device comprising:
a substrate comprising an inlet region and a first storage region;
a fluid transporting channel in fluid communication with the inlet region;
an expandable component in fluid communication with the fluid transporting channel and coupled to a movable arm; and
a fluid transporting region coupled to the movable arm and operable to be moved in a horizontal direction to the fluid transporting channel to thereby form fluidic contact between the inlet region and the first storage region upon expansion of the expandable component.

2. The microfluidic device of claim 1:
wherein the substrate further comprises a second storage region and a third storage region;
wherein the fluid transporting region is operable to be moved in the horizontal direction parallel to the fluid transporting channel to thereby form fluidic contact between the inlet region and the second storage region; and
wherein the fluid transporting region is operable to be moved in the horizontal direction parallel to the fluid transporting channel to thereby form fluidic contact between the inlet region and the third storage region.

3. The microfluidic device of claim 1:
wherein the substrate further comprises one or more additional fluid storage regions; and
wherein the fluid transporting region is operable to be moved in the horizontal direction parallel to the fluid transporting channel to thereby form fluidic contact between the inlet region and each of the one or more additional fluid storage regions.

4. The microfluidic device of claim 1, further comprising a localized dissolvable delay in contact with the fluid transporting channel to control flow rate of a first fluid through the fluid transporting channel.

5. The microfluidic device of claim 4, wherein the localized dissolvable delay is a region comprising a mixture selected from the group consisting of sugar-based compositions, sucrose compositions, fructose compositions, sucrose and fructose compositions, trehalose compositions, glucose compositions, glucose and sucrose compositions, galactose compositions, dextran compositions, isomalt compositions, maltitol compositions, lactitol compositions, soluble macromolecules, water-soluble polymers, polyvinyl alcohol, polyvinyl alcohol compositions, pullulan, pullulan composites, glycerol, polysorbate 20, and combinations thereof.

6. The microfluidic device of claim 5, wherein delay is modulated via a mechanism selected from the group consisting of molecular weight of constituents in the mixture, concentration of the mixture, constituents in the mixture, and combinations thereof.

7. The microfluidic device of claim 5, wherein the delay region is deposited on the fluid transporting channel.

8. The microfluidic device of claim 1, wherein the substrate is selected from the group consisting of paper, cellulose paper, chromatography paper, filter paper, Whatman Grade 1 chromatography paper, Whatman Grade 1 filter paper, Whatman Grade 2 filter paper, Whatman Grade 3 filter paper, Whatman Grade 4 filter paper, Whatman Grade 591 filter paper, Whatman Grade 595 filter paper, Whatman Grade 598 filter paper, Fisherbrand quantitative grade filter paper, Fisherbrand qualitative grade filter paper, nitrocellulose paper, a membrane, Amersham protran nitrocellulose membrane, Whatman fast flow high performance nitrocellulose membrane, immunopore nitrocellulose membrane, and combinations thereof.

9. The microfluidic device of claim 1, wherein the substrate comprises a control line in fluid communication with the fluid transporting channel.

10. The microfluidic device of claim 1, wherein the substrate comprises a test line in fluid communication with the fluid transporting channel.

11. The microfluidic device of claim 1, wherein the fluid transporting channel comprises a first analyte binding agent and the inlet region comprises a second analyte binding agent.

12. A method of fluid flow, the method comprising:
receiving a first fluid at an inlet region on a substrate;
receiving a second fluid at a fluid storage region on the substrate;
flowing the first fluid through a fluid transporting channel on the substrate in fluid communication with the inlet region;
actuating a fluid transporting region coupled to a movable arm operable to be moved in a horizontal direction to the fluid transporting channel via expansion of an expandable component in fluid communication with the fluid transporting channel; and
flowing the second fluid through the fluid transporting channel.

13. The method of claim 12, further comprising delaying flow of the first fluid through the fluid transporting channel via a delay region comprising a mixture selected from the group consisting of sugar-based compositions, sucrose compositions, fructose compositions, sucrose and fructose compositions, trehalose compositions, glucose compositions, glucose and sucrose compositions, galactose compositions, dextran compositions, isomalt compositions, maltitol compositions, lactitol compositions, soluble macromolecules, polymers, polyvinyl alcohol, polyvinyl alcohol compositions, water-soluble polymers, pullulan, pullulan composites, glycerol, polysorbate 20, and combinations thereof.

14. The method of claim 13, wherein the delaying flow of the first fluid is modulated via a mechanism selected from the group consisting of molecular weight of constituents in the mixture, concentration of the mixture, constituents in the mixture, and combinations thereof.

15. The method of claim 12, wherein the fluid transporting channel comprises a first analyte binding agent and the inlet region comprises a second analyte binding agent.

16. The method of claim 15, further comprising:
resuspending the second analyte binding agent in the first fluid;
capturing an analyte in the first fluid with the second analyte binding agent; and
capturing the second analyte binding agent and the analyte with the first analyte binding agent.

17. The method of claim 15, wherein the fluid transporting channel comprises a component capable of binding to the second analyte binding agent.

18. The method of claim 17, further comprising capturing the second analyte binding agent with the component capable of binding to the second analyte binding agent.

19. The method of claim 12, further comprising:
washing the inlet region and the fluid transporting channel with the second fluid; and
removing uncaptured components in the first fluid.

20. The method of claim 12, further comprising reading signal from the fluid transporting channel, wherein the reading is conducted via the group consisting of surface enhanced Raman spectroscopy, colorimetry, absorbance, fluorescence, chemiluminescence, magnetic intensity, and combinations thereof.
